(19) United States
(12) Patent Application Publication
(10) Pub. No.: US 2022/0238221 A1
(43) Pub. Date: Jul. 28, 2022

(54) SYSTEM AND METHOD FOR DEVELOPMENT AND MANAGEMENT OF A TRUSTED NETWORK

(71) Applicant: Seth Feuerstein, New Haven, CT (US)

(72) Inventor: Seth Feuerstein, New Haven, CT (US)

(21) Appl. No.: 17/583,423

(22) Filed: Jan. 25, 2022

Related U.S. Application Data
(60) Provisional application No. 63/141,290, filed on Jan. 25, 2021.

(51) Int. Cl. G16H 40/67; G16H 70/20; A61B 5/16

(52) U.S. Cl. CPC G16H 40/67; A61B 5/165; G16H 70/20

(57) ABSTRACT

A system and method for development and management of a trusted support network for an at-risk person. The system provides a computerized interface providing the at-risk person with both synchronous and asynchronous support, to ensure that the at-risk person is adequately supported at critical times to lessen the risk of harm. The system provides for training of laypersons, so that a layperson can provide effective support and intervention to avert adverse outcomes for such an at-risk person. Further, the system provides for communication among multiple members of a support team. Further still, the system provides for delivery of not only person-to-person supportive communications, but also automated composition and/or delivery of supportive communications. Accordingly, the system can cause display of notices/notifications/alerts and/or placing of telephone calls, sending of text messages, etc. and provide communications in lieu of, or in addition to, those from a supporter.

### CROSS-REFERENCE TO RELATED APPLICATION

This application claims the benefit of priority, under 35 U.S.C. § 119(e), of U.S. Provisional patent application No. 63/141,290, filed Jan. 25, 2021, the entire disclosure of which is hereby incorporated herein by reference.

### FIELD OF THE INVENTION

The present invention relates generally to support and intervention of patients or other persons at-risk for various medical and/or behavioral problems, and more specifically, to a computerized system and method for developing a trusted, curated support network for supporting and intervening to avert adverse outcomes for members of at-risk populations, and for educating, training, and/or supporting members of the network.

### DISCUSSION OF RELATED ART

Medical and behavioral health are key parts of overall health, and can contribute to problems of at-risk populations. For example, the teenager population is generally recognized as a population at risk for drug overdose, suicidality and accidental death.

Clinical patient interactions are performed in a variety of settings in an attempt to measure a person's behavioral status and functional situations across a broad range of clinical domains such as mood, anxiety, psychosis, suicidality, obsessions, compulsions, addictions and medication response for these as well. By way of example, a person arriving at an Emergency Room (ER) of a hospital may be submitted to a clinical patient assessment to screen the patient for suicidality or may present for suicidality.

However, even in the most intensive care settings, assessments are performed on patients infrequently and there are limited options for intervention. Even when an assessment or intervention is performed properly and timely and a relevant condition is identified, and the person is admitted, observed, and treated with medications, it often does not reduce, or adequately reduce, the risk.

Further, the most acute need for an intervention may manifest itself outside of the clinical setting, both for patient and non-patients. Often, a troubled teenager will does not have someone to confide in or if there is someone they might confide in, seek advice from or otherwise engage with they do not know how to identify those people or how to best be in touch with them or those people are young and are not sure how to help. Such contacts may be sympathetic and may wish to help, but they invariably are not aware the teen is in need, are not comfortable in knowing when and how to connect with the teen and/or they are not adequately trained to ensure that the proper care and/or intervention is delivered to the troubled teen.

What is needed is a system and method for supporting and intervening to avert adverse outcomes for members of at-risk populations, particularly outside of the clinical care setting, for both patients in the active care of a medical professional, and those that are not patients.

### SUMMARY

The present invention provides a computerized system and method for developing a trusted support network for supporting and intervening to avert adverse outcomes for members of at-risk populations, and for educating, training, and/or supporting members of the network with and without the assistance of others and/or medical professionals. The system provides a computerized interface providing the at-risk person with both synchronous and asynchronous support, to ensure that the at-risk person is adequately supported at critical times to lessen the risk of harm. The system provides for training of laypersons, so that a layperson can provide effective support and intervention to avert adverse outcomes for such an at-risk person. Further, the system provides for communication among multiple members of a support team. Further still, the system provides for delivery of not only person-to-person supportive communications, but also automated composition and/or delivery of supportive communications. Accordingly, the system can cause display of notices/notifications/alerts and/or placing of telephone calls, sending of text messages, etc. and provide communications in lieu of, or in addition to, those from a supporter. The system further provides for reporting of interventions to support team members and/or caregivers.

### BRIEF DESCRIPTION OF THE FIGURES

For a better understanding of the present invention, reference may be made to the accompanying drawings in which:

FIG. 1 is a system diagram showing an exemplary network computing environment in which the present invention may be employed;

FIG. 2 is a schematic diagram of an exemplary special-purpose Support Team Network Management System computing device in accordance with an exemplary embodiment of the present invention; and

FIGS. 3A and 3B show a flow diagram illustrating an exemplary method involving operation of the Support Team Network Management System in accordance with an exemplary embodiment of the present invention.

### DETAILED DESCRIPTION

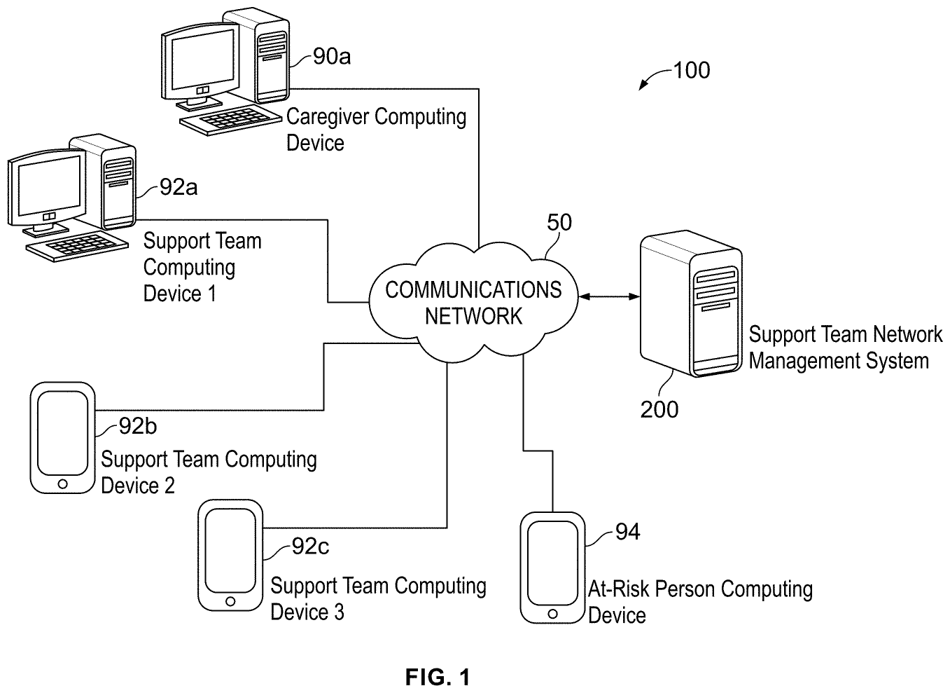

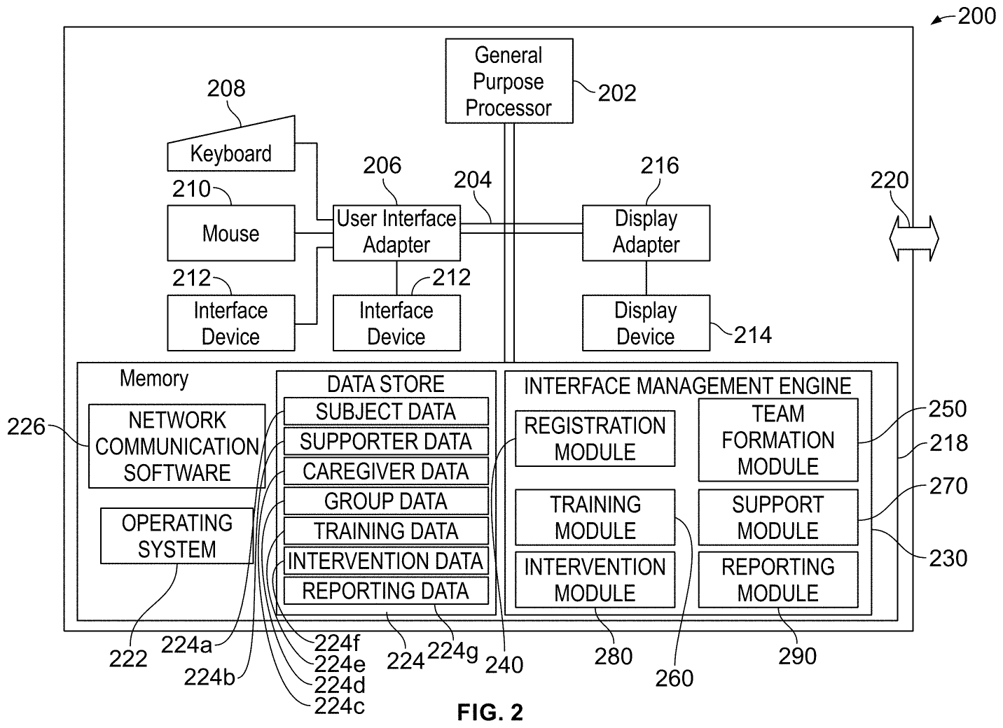

According to illustrative embodiment(s) of the present invention, various views are illustrated in FIGS. 1-2 and like reference numerals are used consistently throughout to refer to like and corresponding parts of the invention for all of the various views and figures of the drawings.

The following detailed description of the invention contains many specifics for the purpose of illustration. Any one of ordinary skill in the art will appreciate that many variations and alterations to the following details are within scope of the invention. Accordingly, the following implementations of the invention are set forth without any loss of generality to, and without imposing limitations upon, the claimed invention.

The present invention provides a system and method for developing a trusted support team support network for supporting and intervening to avert adverse outcomes for members of at-risk populations, and for educating, training, and/or supporting members of the support team network.

### System Environment

An exemplary embodiment of the present invention is discussed below for illustrative purposes. FIG. 1 is a system diagram showing an exemplary network computing environment 100 in which the present invention may be employed. As shown in FIG. 1, the exemplary network environment 100 includes conventional computing hardware and software for communicating via a communications network 50, such as the Internet, etc., using Caregiver Computing Device 90, Support Team Computing Devices 92a, 92b, 92c and/or At-Risk Person Computing Device 94, each of which may be, for example, one or more personal computers/PCs, laptop computers, tablet computers, smartphones, or other computing device hardware.

In accordance with a certain aspect of the present invention, one or more of the Clinician Computing Device 90, the Support Team Computing Devices 92a, 92b, 92c and/or the At-Risk Person Computing Device includes an “app” or other purpose-specific software in accordance with the present invention, although this is not required in all embodiments.

In accordance with the present invention, the network computing environment 100 further includes the Support Team Network Management System (STNMS) 200. In this exemplary embodiment, the STNMS 200 is operatively connected to the Caregiver Computing Device 90, the Support Team Computing Devices 92a, 92b, 92c and the At-Risk Person Computing Device 94 for data communication via the communications network 50. For example, the STNMS 200 may gather user data or other inputs from each device 90, 92a, 92b, 92c, 94 by communication via the communications network 50. Hardware and software for enabling communication of data by such devices via such communications networks are well known in the art and beyond the scope of the present invention, and thus are not discussed in detail herein.

Accordingly, in the event that a troubled teen or other at-risk person seeks to talk to/confide in any person, including a layperson that is not a properly trained medical professional, about suicidality, drug abuse and/or other concerns, the present invention provides a system and method for providing the layperson with experience and tools so that the layperson can provide effective support and intervention to avert adverse outcomes for such an at-risk person. More particularly, the system provides a platform for educating, training, and/or supporting individual laypersons and/or developing a trusted Support Team support network for supporting the at-risk person and intervening to avert an adverse outcome. The present invention provides a specific technology platform for doing so, so that the at-risk person may seek and receive help through not only formal medical/healthcare channels, but also through informal channels involving social contacts and other layperson acquaintances, thus breaking down barriers to delivering adequate support and interventions to at-risk persons, and in particularly, providing technology-based interfaces of a type that are more likely to be used by teenagers, which are a particularly vulnerable class of at-risk persons. In addition the system can cause display of notices/notifications/alerts and/or placing of telephone calls, sending of text messages, etc. to prompt/urge/“nudge” support team members about when to reach out to the teen or at-risk person.

In any case, users, such as teens or other members of the at-risk populations may register with the system and create an account, e.g., as an at-risk subject. In addition members of a clinical team, such as an inpatient unit can suggest the system and enroll the teen as can educational institutions and other organizations where such teens are often identified including pediatricians and university clinics. Additionally, people identified via interactions with the at-risk person or otherwise likely to play the role of supportive Support Team members, such as parents, schoolteachers, college professors, sports coaches, clergy members, friends and/or relatives may also register with the system and create their owner user accounts. Notably, this may be done prospectively, without regarding to any particular at-risk person. Similarly, this may be done retrospectively, with regard to a particularly at-risk person, for example, after the at-risk person has begun communicating to a particular Support Team. Such registration is managed by the Registration Module 240, and at-risk person data is stored as Subject Data, and supportive Support Team data is stored as Support Team Data in the data store 224 of the STNMS.

After registration, the STNMS 200 may provide training content to registered supporters. The training content may be transmitted from the STNMS 200 via the network 50 to a device of a Support Team member, such as Support Team Computing Device 92a, 92b, 92c. The training content may be in the form of instructions, videos, reading materials, a social network of other Support Team individuals, etc. The training content may provide instruction suitable for educating a layperson as to how and when to interact with an at-risk person, so that the layperson can provide better support to the at-risk person. The STNMS 200 may thereby effectively control the Support Team Computing Device 92a, 92b, 92c to cause display of video, text and/or questions and/or other prompts to the layperson, and the layperson may interact with their device 92a, 92b, 92c to provide to the device, in an active fashion, input responsive to the questions/prompts—e.g., by touching a touchscreen, using a stylus, typing on a keyboard, manipulating a mouse, etc. The questions/prompts may be presented based on training content data stored in the memory of the device and/or in the STNMS 200. Such delivery of training content is managed by the Training Module 260, using training group data stored as Training Data in the data store 224 of the STNMS.

In certain embodiments, the information content/questions/prompts are defined in predetermined fashion, based on industry guidelines, thought leader guidance, experienced clinicians, or the like, so that they are consistent with best practices for interacting with an at-risk person. In such embodiments, the sequence of content may be static. In other embodiments, the sequence may be dynamic, such that information content is presented according to predefined logic of the training module, but in a fluid sequence that may vary from person to person or session to session, based on input provided actively by the user.

At some point, before or after the training, a particular layperson may be called upon to provide support to a particular at-risk person. In such a case, the layperson may interact with the STNMS 200 to register the layperson as part of a support group associated with that particular at-risk person. Such group formation is managed by the Team Formation Module 250, and group data associated groups (and group members) with particular at-risk subjects is stored as Group Data in the data store 224 of the STNMS 200.

The group supporting a single at-risk person may include 1 or more supportive Support Teams. The STNMS 200 provides a software interface enabling multiple individual persons of a single group to connect, meet, discuss how to support the at-risk person, share information, consider difficult questions/issues collectively, etc. The interconnection of Support Teams within a single group and communication among those Support Teams is managed by the Support Team Support Module 270. Thus, the STNMS 200 creates an active community of trusted Support Teams that can support the at-risk person.

In certain embodiments, the Support Team Support Module 270 may enable one or more supportive Support Teams, or groups of supportive Support Teams, to connect, meet, discuss how to support at-risk persons, share information, consider difficult questions/issues collectively, etc. across more than one at-risk person. The interconnection of Support Teams across multiple at-risk person support groups and communication among those Support Teams is managed by the Support Team Support Module 270.

In certain embodiments, the supportive Support Team(s) are trained by the STNMS 200 to be able to provide a suitable and effective intervention to support the at-risk person and avert an adverse outcome, such as suicide or drug overdose. The intervention may be delivered in person-to-person communications, may occur outside the STNMS 200 system's platform, e.g., via telephone call or text message, or may occur within, or with the involvement or, the STNMS 200 system. Delivery of an intervention via the STNMS 200 may take the form of a telephone call to, a text message to, or an e-mail message to the at-risk person. Alternatively, delivery of an intervention via the STNMS 200 make take the form of delivery of a video, photograph, text or other information content with the at-risk person, e.g., at the At-Risk Person's Computing Device 94. Such delivery of an intervention is managed by the Intervention Module 240, and at-risk person data is stored as Subject Data, and supportive Support Team data is stored as Support Team Data in the data store 224 of the STNMS.

In certain embodiments, the Intervention Module 280 retrieves one or more pre-defined and pre-stored interventions and/or intervention templates from stored Intervention Data stored in the data store 224 of the STNMS. In certain embodiments, the Intervention Module 280 may cause an automated or partially-automated intervention to be delivered to the At-Risk person, e.g., via transmission from the STNMS 200 to the At-Risk Person's Computing Device 94 via the network 50. For example, the Intervention Module 280 may cause an e-mail or text to be prepared by the STNMS 200 and to be presented to a supporting Support Team, for the supporting Support Team to act, customize and/or approve, such that it will be sent to the At-Risk Person's Computing Device 94. Alternatively, for example, the Intervention Module 280 may cause an e-mail or text to be prepared by the STNMS 200 and to be automatedly sent to the At-Risk Person's Computing Device 94. In certain such embodiments, it may appear to the at-risk person that the e-mail or text was sent from the supporting Support Team personally, even if the intervention was initiated by the STNMS 200 without any affirmative action on the part of the supporting Support Team, which can ensure timely delivery of the intervention to the at-risk person. In certain embodiments, the Intervention Module 280 may initiate a phone call to the At-Risk Person's Computing Device 94, e.g., to deliver a pre-recorded message, to connect the at-risk person with the supporting Support Team, or to connect the at-risk person to a treating clinician, other healthcare professional, or simply another supportive person. By way of example, the STNMS 200 may be configured to prompt or initiate a call between the supporting Support Team and the at-risk person accordingly to a predefined schedule, such that no more than an established minimum amount of time transpires between interpersonal contact/telephone calls.

By way of further example, in certain embodiments, the STNMS 200 is configured to transmit a control signal or other data causing a note to be printed, e.g., by a printer, and to be mailed, e.g., via the US Postal Service, from the supporting Support Team, to the at-risk person. The content of the note may be prepared by the supporting Support Team and captured by the STNMS 200, or alternatively may be prepared by the STNMS 200 (e.g., using a pre-stored template), and then sent appearing to have been sent by the supporting Support Team. Using such pre-defined messages/videos/templates helps to ensure that the at-risk person is receiving appropriate (e.g., clinician-approve) content for the at-risk person.

In certain embodiments, the STNMS 200 is configured to alert a supporting Support Team, or multiple Support Teams in a support group for an at-risk person, and/or a clinician or particular treating clinician for the at-risk individual in the event of certain occurrences. This may occur via data transmissions initiated by the STNMS 200 and delivered via the network 50 to one or more of the Clinician's Computing Device 90 and a Support Team Computing Device 92a, 92b, 92c. Such reporting is managed by the Reporting Module 290, and corresponding reporting data may be stored as Reporting Data in the data store 224 of the STNMS.

### Support Team Network Management System

FIG. 2 is a block diagram showing an exemplary Support Team Network Management System (STNMS) 200 in accordance with an exemplary embodiment of the present invention. The STNMS 200 is a special-purpose computer system that includes conventional computing hardware storing and executing both conventional software enabling operation of a general purpose computing system, such as operating system software 222, network communications software 226, and specially-configured computer software for configuring the general purpose hardware as a special-purpose computer system for carrying out at least one method in accordance with the present invention. By way of example, the communications software 226 may include conventional web server software, and the operating system software 22 may include iOS, Android, Windows, Linux software.

Accordingly, the exemplary STNMS 200 of FIG. 2 includes a general-purpose processor, such as a microprocessor (CPU), 102 and a bus 204 employed to connect and enable communication between the processor 202 and the components of the presentation system in accordance with known techniques. The exemplary presentation system 200 includes a user interface adapter 206, which connects the processor 202 via the bus 204 to one or more interface devices, such as a keyboard 208, mouse 210, and/or other interface devices 212, which can be any user interface device, such as a camera, microphone, touch sensitive screen, digitized entry pad, etc. The bus 204 also connects a display device 214, such as an LCD screen or monitor, to the processor 202 via a display adapter 216. The bus 204 also connects the processor 202 to memory 218, which can include a hard drive, diskette drive, tape drive, etc.

The STNMS 200 may communicate with other computers or networks of computers, for example via a communications channel, network card or modem 220. The STNMS 200 may be associated with such other computers in a local area network (LAN) or a wide area network (WAN), and may operate as a server in a client/server arrangement with another computer, etc. Such configurations, as well as the appropriate communications hardware and software, are known in the art.

The STNMS 200 is specially-configured in accordance with the present invention. Accordingly, as shown in FIG. 2, the STNMS 200 includes computer-readable, processor-executable instructions stored in the memory 218 for carrying out the methods described herein. Further, the memory 218 stores certain data, e.g. in one or more databases or other data stores 224 shown logically in FIG. 2 for illustrative purposes, without regard to any particular embodiment in one or more hardware or software components.

Further, as will be noted from FIG. 2, the STNMS 200 includes, in accordance with the present invention, an Interface Management Engine 230, shown schematically as stored in the memory 218, which includes a number of additional modules providing functionality in accordance with the present invention, as discussed in greater detail below. These modules may be implemented primarily by specially-configured software including microprocessor-executable instructions stored in the memory 218 of the STNMS 200. Optionally, other software may be stored in the memory 218 and and/or other data may be stored in the data store 224 or memory 218.

The exemplary embodiment of the STNMS 200 shown in FIG. 2 includes a Registration Module (RM) 240. The RM 240 is responsible for sending data to cause display of graphical user interface windows at one or more of the Caregiver Computing Device 90a, At-Risk Person Computing Device 94 and/or Support Team Computing Devices 92a, 92b, 92c for transmitting data via the network 50 to cause display of graphical user interface windows at such computing devices for gathering user account information, and for receiving/gathering such information and causing it to be stored in the data store 224 of the STNMS 200 as Subject Data 224a (for the at-risk person), Supporter Data 224b (for the support team members) or Caregiver Data 224c (for the caregiver(s)), as appropriate. For example, the RM 240 may cause display of graphical user interface windows requiring a caregiver to provide the following information as part of registration to create a user account for the caregiver: physician name, practice name, practice location/address, practice contact information, or the name of a relevant social worker, relative, or other supporter, etc. The RM 240 may case display of graphical user interface windows for gathering similar/corresponding information from the at-risk person and support team members.

In accordance with the present invention, the exemplary embodiment of the STNMS 200 shown in FIG. 2 also includes a Team Formation Module (TFM) 250. The TFM 250 is responsible for transmitting data via the network 50 to cause display of graphical user interface windows at a one or more of the Support Team Computing Devices 92a, 92b, 92c and/or the At-Risk Person Computing Device 94 for gathering information that can be used to identify and/or add supporters to the support team for a particular at-risk person. For example, this may be done expressly—for example, by the TFM 250 causing display of a graphical user interface window at the At-Risk Person Computing Device 94 that allows the at-risk person to identify by providing suitable input and/or select one or more supporters from a list of supporters retrieved from the Supporter Data 224b. Alternatively, this may be done implicitly—for example, by the TFM causing display of a graphical user interface window at the At-Risk Person Computing Device 94 that allows the at-risk person to identify organizations (schools, churches, social groups, etc.) or other information/characteristics that the TFM may then use to search the Supporter Data 224b and identify one or more supporters associated with those organizations or other information/characteristics (e.g., as recorded in the Supporter Data). With or without selection/approval/confirmation from the at-risk person, and/or the supporter (e.g., by displaying suitable information to the supporter) the TFM 250 may then add one or more supporters to a support team for the particular at-risk person. Accordingly, the TFM 250 manages information gathering, collection, notification and/or approvals as desired to form a support team for each particular at-risk person, and then stores data reflecting the at-risk person/team relationship as Team Data 224d in the data store 224.

In accordance with the present invention, the exemplary embodiment of the STNMS 200 shown in FIG. 2 also includes a Training Module (TM) 260. The TM 260 prepares the supporter, who may be a layperson, to be adequately equipped to provide effective support and/or intervention for the at-risk person/persons that he/she is supporting. More particularly, the TM 260 is responsible for transmitting data via the network 50 to cause display of graphical user interface windows at one or more Support Team Computing Devices 92a, 92b, 92c for displaying and/or gathering information providing suitable training to the associated support team member. For example, the TM 260 may transmit data providing hyperlinks to text, videos and/or other information content suitable for training a support team member to provide suitable counseling and support to the at-risk person. This reduces the risk to the at-risk person.

Suitable information content and/or data identifying such content may be retrieved and stored from the Training Data 224e stored in the data store 224. The TM 260 may also be responsible for selecting a subset of the Training Data 224e that is particularly tailored to each supporter, so that the training experience is customized to each supporter. By way of example, this may be performed by retrieving data from the Supporter Data 224b and processing it according to predetermined logic to match particular information content to the supporter. By way of additional example, the TM 260 may also be responsible for selecting a subset of the Training Data 224e that is particularly tailored to each supporter according to the needs of the particular at-risk person or persons being supported by that supported, so that the training experience is customized to each supporter. By way of example, this may be performed by retrieving data from the Subject Data 224a and/or Team Data 224d, and processing it according to predetermined logic to match particular information content to the supporter. Such training data may be very fluid and dynamic and change frequently over time, e.g., as the supporter data and/or subject data and/or team data changes.

In accordance with the present invention, the exemplary embodiment of the STNMS 200 shown in FIG. 2 also includes a Support Module (SM) 270. The SM 270 manages delivery of support to the at-risk persons. More particularly, the SM 270 is responsible for transmitting data via the network 50 to cause display of graphical user interface windows at one or more Support Team Computing Devices 92a, 92b, 92c for displaying information prompting the support team member to provide support. For example, this may be a prompt to call the at-risk person, or to send the at-risk person a text message, an e-mail, supportive information content, to engage the at-risk person in a chat-like or other system-based communication session, etc. For example, this may be done in response to the at-risk person's activity, such as an express request for support, or activities monitored by the system suggesting a need for support, as may be detected by the Support Module, such as activities or inactivity of the at-risk person, the passage of a minimum amount of time since a last interaction with the at-risk person, etc. In this manner, the STNMS 200 provides a computerized platform for facilitating a human supporter's delivery of timely support to the at-risk person. The SM may use machine learning (ML) and/or artificial intelligence (AI) technologies to schedule meetings or other interactions between the Support Team members and at-risk persons, to prompt the Support Team member to schedule meetings/communications/other interactions with at-risk persons, and/or deliver to the Support Team member suggested phrases/language or other information content (retrieved from the system) for use by the Support Team member in meetings/communications/other interactions with at-risk persons.

Additionally, the SM 270 may identify the various members of the support team for a particular at-risk person, e.g., by retrieving data from the Team Data 224c and/or Supporter Data 224b, and provide a communication platform for communication and information sharing among the various members of that support team. More particularly, the SM 270 may transmit data via the network 50 to cause display of graphical user interface windows at one or more Support Team Computing Devices 92a, 92b, 92c for displaying information prompting the support team member to have a group conference call, to email each other, to complete questionnaires, or to otherwise share observations and other information that may be relevant to support of the team. Further, this provides a mechanism permitting each individual support team member to share ideas with the others, and receive suggestions and/or feedback as to how to be an effective support team member

The SM 270 may also retrieve data from the Subject Data 224a and/or Caregiver Data 224c and transmit data via the network 50 to cause display of graphical user interface windows at the Caregiver Computing Device 90 alerting the Caregiver to interactions between the at-risk person and/or the support team embers, and/or prompting the Caregiver to provide support. For example, this may be a prompt to call the at-risk person, or to send the at-risk person a text message, an e-mail, supportive information content, to engage the at-risk person in a chat-like or other system-based communication session, etc. In this manner, the system provides a computerized platform for facilitating a human caregiver's delivery of timely support to the at-risk person.

In accordance with the present invention, the exemplary embodiment of the STNMS 200 shown in FIG. 2 also includes an Intervention Module (IM) 280. The IM 280 manages delivery of automated interventions to the at-risk persons. More particularly, the IM 280 is responsible for transmitting data via the network 50 to provide a supportive intervention for the at-risk person. For example, the IM 280 may transmit data via the network 50 to cause display of graphical user interface windows at the At-Risk Person Computing Device 94 for displaying information to the at-risk person that provides a supportive intervention. For example, this may be an automated telephone call to the at-risk person, to deliver generic pre-recorded content or customized/personalized pre-recorded content (e.g., from a member of the at-risk person's support team) retrieved from the Intervention Data 224f in the data store 224. By way of alternative example, the IM 280 may transmit data via the network to cause sending of a text message, e-mail, and/or supportive information content and/or to engage the at-risk person in a chat-like or other system-based communication session, etc. to the at-risk person, e.g., by retrieving data from the Intervention Data 224. The content of such a text, e-mail message, etc. may be generated by the system, e.g., using information retrieved from memory, and may be generated programmatically and/otherwise or automatedly by the system. By way of alternative example, the IM 280 may transmit data via the network to cause a computer printer to print a note/letter to the at-risk person, and to cause it to be mailed or otherwise be delivered to the at-risk person. Notably, these messages, etc. may be crafted to appear to come from the STNMS 200/system/service, or perhaps more effectively, to have been sent/provided from a particular member of the support team network, although it may originate with the STNMS 200. In this manner, the STNMS 200 provides a computerized platform for providing a computerized delivery of timely support to the at-risk person, without the need for human intervention. This can be particularly helpful, for example, when the human support team member is temporarily unavailable at a time of need of the at-risk person.

The exemplary embodiment of the STNMS 200 shown in FIG. 2 also includes a Reporting Module (RM) 290. The Reporting Module (RM) 290 is responsible for transmitting data via the network 50 to cause display of appropriate graphical user interface windows (or other messages), or other data to alert and/or inform relevant parties of relevant occurrences. For example, the RM 290 may notify a caregiver and/or parents of a minor child as to the occurrence of various events occurring within the system, such as registering with the system, adding a support team member, asking for support, receiving support via a support team member or via the IM 280, etc. The system may be configured to provide any suitable reporting in response to various events/occurrences, as desired.

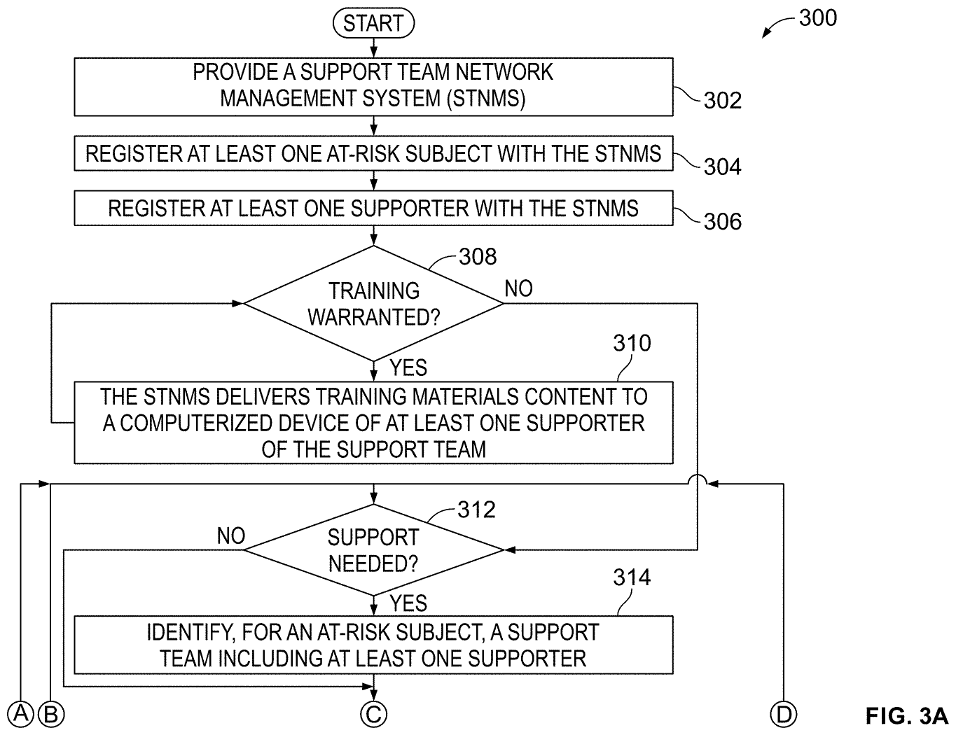

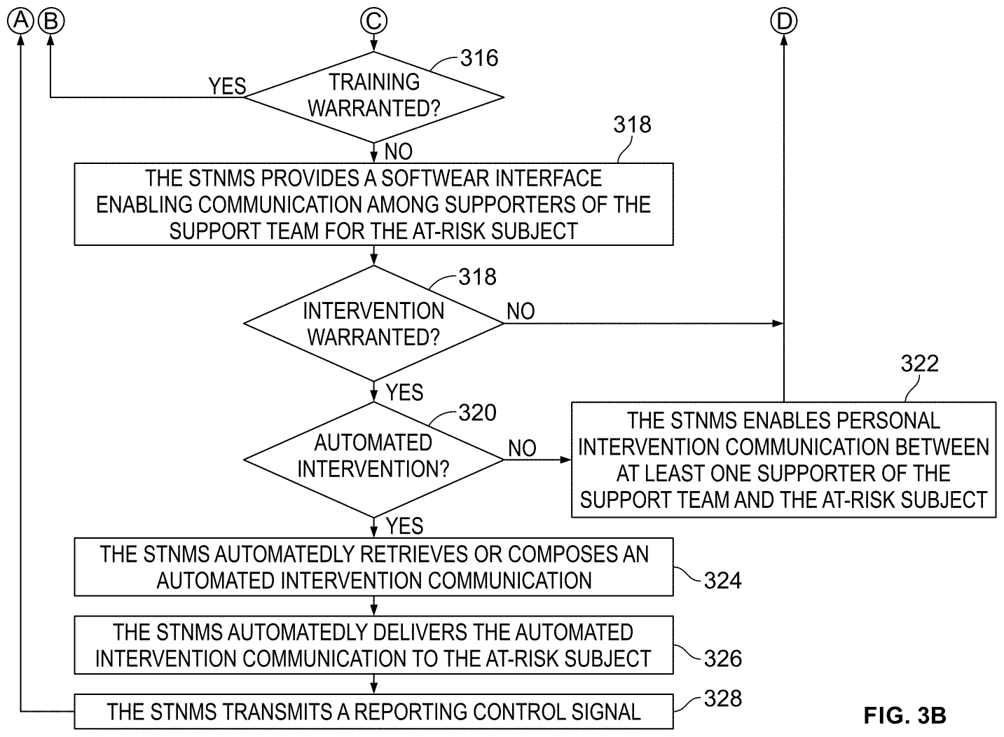

FIGS. 3A and 3B show a flow diagram 300 illustrating an exemplary method involving operation of the STNMS 200 in accordance with an exemplary embodiment of the present invention. Referring now to FIG. 3A, the exemplary method begins with providing a Support Team Network Management System (STNMS) 200 in accordance with the present invention, as shown at 302.

Although the illustrated steps need not take place in the order shown, the exemplary method next involves registering at least one at-risk subject with the STNMS 200, as shown at 304. This may involve, for example, operation of the At-Risk Person Computing Device 94 to conduct data communication/information exchange with the STNMS 200 via the communications network 50, e.g., to provide name, contact information (such as email address, phone number, SMS/texting address, etc.) and information usable to identify potential support team members for the at-risk person, e.g., such as school information, club information, geographical information and other association-type information.

Although the illustrated steps need not take place in the order shown, the exemplary method next involves registering at least one supporter with the STNMS 200, as shown at 304. This may involve, for example, operation of a Caregiver Computing Device 90 and/or a Support Team Computing Devices 92a, 92b, 92c to conduct data communication/information exchange with the STNMS 200 via the communications network 50, e.g., to provide name, contact information (such as email address, phone number, SMS/texting address, etc.) and information usable to identify potential for acting as a support team member for an at-risk person, e.g., such as school information, club information, geographical information and other association-type information.

Referring again to FIG. 3A, the method next involves determining whether training is warranted, as shown at 308. This may be determined in any suitable fashion. For example, a layperson may be required to complete a certain set of educational learning modules, while a professional caregiver may be required to complete certain other educational learning modules. Alternatively, for example, educational learning modules may be required to be complete over time, according to a predefined or undefined schedule. If it is determined, according to any suitable logic, that training is warranted, e.g., for a particular supporter, then the STNMS 200 delivers training materials associated with a corresponding educational learning module to a computerized device of that supporter, as shown at 310, and the process flow loops back to determine whether additional training is warranted, as shown at 308. This may allow for pre-training of a supporter, prior to a need to provide support to an at-risk person, for example.

The STNMS 200 may deliver training materials associated with a corresponding educational learning module to a computerized device of a supporter in any suitable fashion. By way of example, the STNMS 200 may do so by transmitting data via the network to a Support Team Computing Device 92a, etc., providing a hyperlink to text, videos and/or other information content suitable for training a support team member, e.g., using contact information for the supporter gathered for that supporter during the registration process, and stored by the STNMS 200.

In the exemplary method, if it is determined that additional training is not warranted at step 308, it is next determined whether support is needed for an at-risk person, as shown at 312. If support is not needed, then it may be determined whether additional training is warranted for a supporter, as shown at 316, and flow may continue to 312 where it is again determined whether support for an at-risk person is needed, as shown at 312. This additional training may allow for supplemental/ongoing training of a supporter over time, or as particular support needs and associated training arise, for example.

The determination of a need for support at 312 may be determined in any suitable manner. By way of example, it may be determined by receipt at the STNMS 200 of a request for support, e.g., via a data transmission from the At-Risk Person's Computing Device 94 to the STNMS 200, for a request for support, which may be detected by the STNMS 200 directly. Alternatively, for example, a troubled teen or other at-risk person may seek to talk to/confide in a person or otherwise request report from a person directly, outside of the STNMS 200, e.g., by a direct telephone call, text or in-person request between the parties. In this case, the need for support may not be detectable by the STNMS 200 directly, and in this case the STNMS 200 may detect the need for support as a result of a reporting of the need to the system STNMS 200 by data communication to the STNMS 200, or as part of an unregistered person's action to register with the STNMS 200 as a supporter, etc.

If it is determined at 312 that support is needed for at at-risk subject, then the STNMS 200 identifies for the at-risk subject a support team including at least one supporter, as shown at 314. The supporters for the support team for a particular at-risk subject may be identified in any suitable manner. By way of example, people identified via interactions with the at-risk person or otherwise likely to play the role of supportive Support Team members, such as parents, schoolteachers, college professors, sports coaches, clergy members, friends and/or relatives may be identified, e.g., using information gathered from such persons during the registration process with the STNMS 200. Support team members and/or the at-risk subject may also submit to the STNMS 200 a request to be add a particular support person to a support team for a particular at-risk subject, and the STNMS 200 will process those requests accordingly.

In the exemplary method shown in FIG. 3A, if it is determined that support is needed at 312 and that no training is warranted at 316, then the STNMS 200 provides a software interface enabling communication among supporters of the support team for the at-risk subject, as shown at 318, FIG. 3B. More particularly, the STNMS 200 provides a software interface enabling multiple individual persons of a single group to connect, meet, discuss how to support the at-risk person, share information, consider difficult questions/issues collectively, etc., e.g. using contact information gathered during registration and/or provided by the STNMS 200, such as a chat platform, texting platform, e-mail platform and/or audio and/or video conference platform. Thus, the STNMS 200 creates an active community of trusted Support Teams that can support the at-risk person.

In the exemplary method shown in FIG. 3B, it is next determined if an intervention is warranted, as shown at 318. The determination of whether an intervention is warranted may be determined in any suitable manner. By way of example, the STNMS 200 may determine whether an intervention is warranted by automated analysis of communications originating from the at-risk person's computing device 94, e.g., by detecting key words in texts/emails suggesting an intervention is warranted, or by virtue of an attempt to communicate with a member of the at-risk person's support team, etc. If it is determined at 318 that an intervention is not warranted, then the flow continues to 312 and 316 where it is again determined if support and training are needed, as described above.

If, however, it is determined at 318 that an intervention is warranted, then it is determined at 320 if an automated intervention is warranted, as shown at 320. This determination may be made in any suitable manner. For example, it may be determined that an automated intervention is not warranted because a member of the at-risk person's support team may transmit a data communication to the STNMS 200 indicating that, in the member's judgment, no intervention is warranted. By way of alternative example, it may be determined that an automated intervention is not warranted because a member of the at-risk person's support team may transmit a data communication to the STNMS 200 indicating that the member is going to (or has) personally contact/communicate with the at-risk person to deliver an intervention. By way of further alternative example, the STNMS 200 may automatedly determining that an automated intervention is not warranted by detecting the conducting of an intervention communication between the at-risk person and the member of the at-risk person's support team by way of the communication platform enabled by the STNMS 200. In either alternative case, the STNMS 200 enables a personal (person-to-person) intervention communication between at least one supporter of the support team and the at-risk subject, as shown at 322. By way of example, the intervention communication may be enabled by the STNMS 200 enabling communication via the communication platform enabled by the STNMS 200, or by the STNMS 200 providing a notification and/or contact information to the supporter for communication with the at-risk person outside of the communication platform enabled by the STNMS 200. In this exemplary embodiment, flow then returns to 312 for further support, training and/or interventions as described above.

If, however, it is determined at 320 that an automated intervention is warranted, then the STNMS 200 retrieves or composes an automated intervention communication, as shown at 324. For example, a previously-stored communication prepared by a support team member for the at-risk person may be retrieved, or a generic/stock pre-stored communication available within the system may be combined with appropriate at-risk person recipient information (e.g., retrieved from the at-risk person's registration contact information) and with appropriate support team supporter member information (e.g., retrieved from the supporter's registration contact information).

The STNMS 200 then delivers the automated intervention communication top the at-risk person, as shown at 326. The intervention communication may be delivered to the at-risk person via any suitable communication platform, e.g., as a pre-recorded audio message delivered to the at-risk person by a call to the at-risk person's telephone, by sending of a text message or email containing relevant interventional message content to the at-risk person's computing device 94, or by sending a URL or hyperlink to an audio, video or other content to the at-risk person's computing device 94, e.g., using contact information gathered for the at-risk person during the registration process. In this manner, a customized and/or personalized intervention communication may be provided by the STNMS 200 to the at-risk person promptly at a time of need, even if one or all supporters of the at-risk person's support team are unavailable, unreachable, or slow to respond.

In this example, the STNMS 200 then transmits a control signal or other data for reporting delivery of the intervention to the at-risk person, to alert a supporter, supporting Support Team, or multiple Support Teams in a support group for an at-risk person, and/or a clinician or particular treating clinician for the at-risk individual in the event of certain occurrences, as shown at 328, and the method flow returns to 312. This may occur via data transmissions initiated by the STNMS 200 and delivered via the network 50 to one or more of the Clinician's Computing Device 90 and a Support Team Computing Device 92a, 92b, 92c.

Accordingly, it will be appreciated that the present invention provides a computerized system and method for development and management of a trusted support network for an at-risk person, and that the STNMS 200 provides a computerized interface providing the at-risk person with both synchronous and asynchronous support, from both laypersons and professional caregivers, while helping to avoid dangerous isolation of the at-risk person, and to ensure that the at-risk person is adequately supported at critical times, to lessen the risk of harm to the at-risk person. Further, it will be appreciated that even a layperson that is not a medical professional properly trained to provide appropriate support to an at-risk person in relation to suicidality, drug abuse and/or other concerns, is hereby provided with a system and method for providing the layperson with experience and tools so that the layperson can provide effective support and intervention to avert adverse outcomes for such an at-risk person. More particularly, the system provides a platform for educating, training, and/or supporting individual laypersons and/or developing a trusted Support Team support network for supporting the at-risk person and intervening to avert an adverse outcome. The present invention provides a specific technology platform for doing so, so that the at-risk person may seek and receive help through not only formal medical/healthcare channels, but also through informal channels involving social contacts and other layperson acquaintances, thus breaking down barriers to delivering adequate support and interventions to at-risk persons, and in particularly, providing technology-based interfaces of a type that are more likely to be used by teenagers, which are a particularly vulnerable class of at-risk persons. In addition the system can cause display of notices/notifications/alerts and/or placing of telephone calls, sending of text messages, etc. to prompt/urge/“nudge” support team members about when to reach out to the teen or at-risk person.

The various implementations and examples shown above illustrate a method and system for development and management of a trusted network using an electronic device. As is evident from the foregoing description, certain aspects of the present implementation are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present implementation. Accordingly, the specification and drawings are to be regarded in an illustrative rather than a restrictive sense.

Certain systems, apparatus, applications or processes are described herein as including a number of modules. A module may be a unit of distinct functionality that may be presented in software, hardware, or combinations thereof. When the functionality of a module is performed in any part through software, the module includes a computer-readable medium. The modules may be regarded as being communicatively coupled. The inventive subject matter may be represented in a variety of different implementations of which there are many possible permutations.

The methods described herein do not have to be executed in the order described, or in any particular order. Moreover, various activities described with respect to the methods identified herein can be executed in serial or parallel fashion. In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separate embodiment.

In an exemplary embodiment, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a server computer, a client computer, a personal computer (PC), a tablet PC, a set-top box (STB), a Personal Digital Assistant (PDA), a cellular telephone, a smart phone, a web appliance, a network router, switch or bridge, or any machine capable of executing a set of instructions (sequential or otherwise) that specify actions to be taken by that machine or computing device. Further, while only a single machine is illustrated, the term “machine” shall also be taken to include any collection of machines that individually or jointly execute a set (or multiple sets) of instructions to perform any one or more of the methodologies discussed herein.

The example computer system and client computers include a processor (e.g., a central processing unit (CPU) a graphics processing unit (GPU) or both), a main memory and a static memory, which communicate with each other via a bus. The computer system may further include a video/graphical display unit (e.g., a liquid crystal display (LCD) or a cathode ray tube (CRT)). The computer system and client computing devices also include an alphanumeric input device (e.g., a keyboard or touch-screen), a cursor control device (e.g., a mouse or gestures on a touch-screen), a drive unit, a signal generation device (e.g., a speaker and microphone) and a network interface device.

The system may include a computer-readable medium on which is stored one or more sets of instructions (e.g., software) embodying any one or more of the methodologies or systems described herein. The software may also reside, completely or at least partially, within the main memory and/or within the processor during execution thereof by the computer system, the main memory and the processor also constituting computer-readable media. The software may further be transmitted or received over a network via the network interface device.

The term “computer-readable medium” should be taken to include a single medium or multiple media (e.g., a centralized or distributed database, and/or associated caches and servers) that store the one or more sets of instructions. The term “computer-readable medium” shall also be taken to include any medium that is capable of storing or encoding a set of instructions for execution by the machine and that cause the machine to perform any one or more of the methodologies of the present implementation. The term “computer-readable medium” shall accordingly be taken to include, but not be limited to, solid-state memories, and optical media, and magnetic media.

What is claimed is:

1. A computer-implemented method for developing and managing a trusted support network via a computerized support team network management system having at least one processor and a memory operatively coupled to the memory and storing instructions executable by the processor, the method comprising:
registering at least one at-risk subject with the support team network management system, said registering comprising gathering personal information associated with said at least one at-risk subject;
registering at least one supporter with the support team network management system, said registering comprising gathering personal information associated with said at least one supporter;
determining whether training is warranted for said at least one supporter, and if training is warranted, then delivering training materials associated with a corresponding educational learning module to a computerized device of said at least one supporter;
determining whether support is needed for an at-risk subject; and
if it is determined that support is needed, then:
identifying for said at-risk subject a support team including at least one supporter;
determining if an intervention for said at-risk person is warranted; and
delivering an intervention communication to the at-risk person if it is determined that an intervention is warranted.

2. The computer-implemented method of claim 1, further comprising:
reporting delivery of said intervention communication to at least one of a supporter, a support team comprising the supporter, and a caregiver for the at-risk person.

3. The computer-implemented method of claim 1, wherein said reporting delivery of said intervention communication comprises transmission of data via a communications network.

4. The computer-implemented method of claim 1, further comprising:
enabling communication among a plurality of supporters of said support team for said at-risk subject.

5. The computer-implemented method of claim 4, wherein said enabling communication among a plurality of supporters of said support team for said at-risk subject comprises providing a software interface enabling communication among said plurality of supporters.

6. The computer-implemented method of claim 4, wherein said enabling communication among a plurality of supporters of said support team for said at-risk subject comprises providing to at least one of said plurality of supporters access to contact information of at least one other of said plurality of supporters.

7. The computer-implemented method of claim 1, wherein said registering said at least one at-risk subject comprises gathering personal information comprising at least one of contact information and association-type information associated with said at least one at-risk subject.

8. The computer-implemented method of claim 1, wherein said registering said at least one supporter comprises gathering personal information comprising at least one of contact information and association-type information associated with said at least one supporter.

9. The computer-implemented method of claim 1, wherein said determining whether training is warranted for said at least one supporter comprises determining whether said at least one supporter is a layperson.

10. The computer-implemented method of claim 1, wherein said determining whether training is warranted for said at least one supporter comprises determining a time period since a last completion of training for said at least-one supporter.

11. The computer-implemented method of claim 1, wherein said delivering of training materials comprises transmitting, via a network to the computerized device of said at least one supporter, data comprising at least one of a text file, an image file, an audio file, a video file and a hyperlink to any one of same.

12. The computer-implemented method of claim 1, wherein said delivering of training materials comprises transmitting data, via the network to the computerized device of said at least one supporter, using contact information gathered during registering said at least one support with the support team network management system.

13. The computer-implemented method of claim 1, wherein said determining whether support is needed comprises receiving, at the support team network management system, a data communication from said at-risk subject's computerized device, said data communication comprising a request for support from said at-risk subject.

14. The computer-implemented method of claim 1, wherein said determining whether support is needed comprises receiving, at the support team network management system, a data communication from a supporter's computerized device reporting the at-risk subject's request for support.

15. The computer-implemented method of claim 1, wherein said identifying for said at-risk subject said support team including at least one supporter comprises identifying at least one supporter having a common characteristic with said at-risk person.

16. The computer-implemented method of claim 15, wherein said identifying at least one supporter having a common characteristic with said at-risk person comprises comparing personal information of said at-risk subject to personal information of supporters registered with said support team network management system to identifying commonality of at least one of a family, a school, a university, a sports team, and a religious institution.

17. The computer-implemented method of claim 15, wherein said identifying at least one supporter having a common characteristic with said at-risk person comprises referencing personal information gathered during registration to identify a supporter having requested to serve as a supporter for said at-risk person.

18. The computer-implemented method of claim 1, wherein said determining if an intervention for said at-risk person is warranted is determined by the support team network management system performing an automated analysis of a communication originating from said at-risk person's computing device

19. The computer-implemented method of claim 18, wherein said performing an automated analysis of a communication originating from said at-risk person's computing device comprises detecting key words suggesting an intervention is warranted.

20. The computer-implemented method of claim 1, wherein said determining if an intervention for said at-risk person is warranted is determined by the support team network management system identifying an attempt by the at-risk person to communicate with a supporter of said at-risk person's support team.

21. The computer-implemented method of claim 1, wherein said delivering an intervention if it is determined that an intervention is warranted comprises composing and transmitting an automated intervention communication providing support to said at-risk subject.

22. The computer-implemented method of claim 21, wherein said composing and transmitting an automated intervention communication providing support to said at-risk subject comprises retrieving from memory a previously-stored communication prepared by a support team member for the at-risk person.

23. The computer-implemented method of claim 21, wherein said composing and transmitting an automated intervention communication providing support to said at-risk subject comprises retrieving from memory a previously-stored generic communication and combined it with at least one of at-risk subject recipient information and support team supporter member information.

24. The computer-implemented method of claim 1, wherein said delivering an intervention if it is determined that an intervention is warranted comprises delivering at least one of a pre-recorded audio message delivered to the at-risk person by telephone call, a text message delivered to the at-risk person's computing device, an e-mail message delivered to the at-risk person's computing device.

25. The computer-implemented method of claim 1, wherein said delivering an intervention if it is determined that an intervention is warranted comprises the support team network management system facilitating a personal (person-to-person) intervention communication between at least one supporter of said support team and said at-risk subject.

26. A support team network management system comprising:
a processor;
a memory operatively connected to the processor, said memory storing executable instructions that, when executed by the processor, causes the network management system to perform a method for developing and managing a trusted support network;
a registration module comprising a first set of executable instructions stored in said memory for causing display of a graphical user interface configured for gathering user account information for at least one subject and at least one supporter;
a team formation module comprising a second set of executable instructions stored in said memory for adding at least one of said at least one supporter to a support team for at least one of said at least one subject;
a training module comprising a third set of executable instructions stored in said memory for causing display of a graphical user interface configured for causing display of training information content to said at least one of said at least one supporter; and
a support module comprising a fourth set of executable instructions stored in said memory for causing display of a graphical user interface configured for displaying information content prompting said at least one of said at least one support to communicate with said at least one of said at least one subject.

27. The support team network management system of claim 1, further comprising:
an intervention module comprising a fifth set of executable instructions stored in said memory and executable to provide a supportive communication to said at least one of said at least one subject.

28. The support team network management system of claim 1, further comprising:
a reporting module comprising a sixth set of executable instructions stored in said memory and operative to provide reporting of delivery of a supportive communication to at least one of a supporter, a support team comprising the supporter, and a caregiver for the at-risk person.

29. A computer program product for implementing a method for developing and managing a trusted support network, the computer program product comprising a non-transitory computer-readable medium storing executable instructions that, when executed by a processor, cause a computerized system to perform a method for support team network management, the method comprising:
registering at least one at-risk subject with aa support team network management system, said registering comprising gathering personal information associated with said at least one at-risk subject;
registering at least one supporter with the support team network management system, said registering comprising gathering personal information associated with said at least one supporter;
determining whether training is warranted for said at least one supporter, and if training is warranted, then delivering training materials associated with a corresponding educational learning module to a computerized device of said at least one supporter;
determining whether support is needed for an at-risk subject; and
if it is determined that support is needed, then:
identifying for said at-risk subject a support team including at least one supporter;
determining if an intervention for said at-risk person is warranted; and
delivering an intervention communication to the at-risk person if it is determined that an intervention is warranted.
